(19) United States
(12) Patent Application Publication
(10) Pub. No.: US 2009/0186411 A1
(43) Pub. Date: Jul. 23, 2009

(54) Cell culture apparatus, method for producing the apparatus and cell culture method

(75) Inventors: Bernd Hoffmann, Juelich (DE); Bodo Borm, Bonn (DE); Rudolf Merkel, Juelich (DE)

Correspondence Address: JORDAN AND HAMBURG LLP, 122 EAST 42ND STREET, SUITE 4000, NEW YORK, NY, 10168, US

(73) Assignee: Forschungszentrum Juelich GmbH, Juelich (DE)

(21) Appl. No.: 12/308,789

(22) PCT Filed: Jun. 14, 2007
(86) PCT No.: PCT/DE2007/001054

§ 371 Date: Dec. 23, 2008

(30) Foreign Application Priority Data
Jun. 24, 2006 (DE) 10 2006 029 051.8

(51) Int. Cl. C12N 5/06; C12N 5/00; C12M 1/00; B05D 3/00

(52) U.S. Cl. 435/377; 435/396; 435/307.1; 435/305.1; 435/287.2; 427/2.11

(57) ABSTRACT

The invention relates to a cell culture apparatus for cells, characterized by a surface composed of an unstructured elastomer. It is thus possible for cells to be cultured under close to natural conditions in relation to their environmental elasticity. A method for producing an apparatus according to the invention is disclosed, as is a cell culture method using such an apparatus.

### BACKGROUND OF THE INVENTION

The invention relates to a cell culture apparatus, a method for producing the apparatus, and to a cell culture method performed using such an apparatus.

Cell culture vessels and apparatuses made of plastics, such as polypropylene, polystyrene, or glass, are known from the state of the art. These vessels or apparatuses are available in a wide variety of configurations and have been used routinely for many years, for example in the form of cell culture bottles, test tubes, multi-well plates, Petri dishes and the like.

Polypropylene and polystyrene are thermoplastics. These substances are physiologically safe and are allowed for food packaging without restrictions.

Types of glass used for the cell culture are, for example, soda-lime glass or borosilicate glass. Due to the optical properties and the excellent resistance to chemicals and high temperatures thereof, glass is widely used for laboratory applications. These surfaces allow the culture of cells, without absolutely necessitating any additional chemical modification of the surface.

The disadvantage is that the types of glass and plastics mentioned above do not allow any long-term analysis or culture of cells under in vivo-equivalent conditions.

Despite this significant disadvantage, they are routinely employed in many scientifically and economically relevant experiments, such as active ingredient screening or in cancer research.

From Discher et al. (Discher D. E., Janmey P., Yu-Li Wang (2005). Tissue cells feel and respond to the stiffness of their substrate. Science, 310, 1139-1143), it is known that tissue cells can respond to the stiffness of the substrates thereof.

A disadvantage is that also the known substrates are not suitable for long-term cell cultures and for storage or retention.

It is the object of the invention to provide a cell culture apparatus, which allows for culture of cells under in vivo-like conditions.

### SUMMARY OF THE INVENTION

The object is achieved by an apparatus and a method for the production thereof which includes an apparatus support structure, and a layer or film comprised of an elastomer carried on said apparatus support structure, the later or film having an unstructured surface for receiving cells.

According to the invention, the cell culture apparatus is characterized by an unstructured surface comprising an elastomer. Accordingly, the cell culture apparatus has a planar surface comprising an elastomer.

It was recognized as part of the invention that the elastic property of a cell culture surface, expressed in physical terms by the Young modulus, is of critical importance in the culture of cells under in vivo-equivalent conditions. The Young modulus is also referred to as the elastic modulus or the modulus of elasticity.

The Young modulus of elastomers is defined, and can be reproducibly adjusted, depending on types starting substances and production methods.

Advantageously, elastomers are much better suited for the culture of cells than the cell culture vessels used according to the state of the art, which have hard surfaces. In this connection, the chemical composition of the elastomer is less important than the physical property, expressed in form of the Young modulus.

It was found that the elastic surface can be referred to as nearly natural with respect to the softness and elasticity thereof. Advantageously, the elastomer allows the cells to be cultured under virtually in situ or in vivo-equivalent conditions.

As part of the invention it was also found that the hardness of the materials frequently used according to the state of the art, such as polypropylene or polystyrene, is much too high, having a modulus of elasticity of 10⁹ Pa, for example. With respect to elasticity, these materials cannot be referred to as nearly natural. The same also applies to soda-lime glass (modulus of elasticity 7.3*10¹⁰ Pa, Poisson's number: 0.22) and borosilicate glass (modulus of elasticity 6.3*10¹⁰ Pa, Poisson's number: 0.2), which are likewise frequently used.

In addition, it was found as part of the invention that the previously employed culture conditions and surfaces of cell culture apparatuses are unlike the natural environment of cells in terms of stiffness and hardness. Particularly, the cells of multicellular organisms are in contact with considerably softer and more elastic surfaces than those provided by the glass, or the types of plastic, used in the laboratory.

The surface of the apparatus according to the invention has a layer or a film comprising unstructured elastomer for receiving cells. For culture purposes, the cells are brought in contact with and adhere to this surface, either directly or via a layer that promotes culture.

Such an elastomer layer or film accordingly has a planar unstructured surface. The layer, or the film, has no (micro-)structures of any kind.

It was found, as part of the invention, that structures, such as troughs or island-like elevations, are recognized by many cell types and thus disadvantageously prevent the cells from uninfluenced development and reproduction and/or propagation by cell division. A planar surface comprising an elastomer thus advantageously supports the natural development and reproduction of the cells.

Advantageously, the surface of the unstructured elastomer is, for example, at least 1 cm². This can be equated approximately to the surface of a cover slip.

The idea according to the invention in its broadest embodiment encompasses a cell culture apparatus comprising an unstructured elastomer as the surface for the cells, wherein any elastomer that is chemically resistant and biocompatible for the cells employed can be used as the surface of the cell culture apparatus. The elastomer used should advantageously be reproducibly preparable with respect to the elasticity of same.

The unstructured elastomer covers the part of the surface of the cell culture apparatus that is intended to receive the cells and has the form of a layer, which is configured, for example, as a thin film. The elastomer thus forms the substrate for the cells.

In one embodiment of the invention, the elastomer is transparent.

In this way, the cells which may adhere to the elastomer and the progress of the cell culture are easy to observe.

Since the material of the cell culture vessel as such, which is to say without the elastomer, is also generally transparent, close observation of the cells is advantageously possible through the outside wall of the apparatus and through the elastomer surface on the inside of the apparatus.

To this end, in a further advantageous embodiment of the invention, the surface of the apparatus comprising the elastomer has a refractive index in a range of approximately 1.3 to 1.5, and particularly a refractive index of 1.4.

The apparatuses according to the invention, along with the elastomer, are particularly suited to receive adhering cells, which includes more than 95% of all known animal cell types.

The Young modulus of the elastomer in the apparatus according to the invention can be adjusted to between 1 MPa and less than 1 kPa, depending on the experiment.

Elastomer elasticity of the as little as 500 Pa is reproducibly adjustable.

The apparatus according to the invention advantageously comprises an elastomer having a Poisson's ratio, or a Poisson's number, in the range of approximately 0.3 to 0.5. A Poisson's ratio of 0.5 characterizes the elastomer as incompressible, or of constant volume. Mathematically and physically, it can be precisely detected.

In a particularly advantageous embodiment of the invention, the surface of the apparatus comprises an elastomer that is not water-based.

The elastomer on the surface of the apparatus according to the invention is then advantageously maintained lastingly and has an accordingly high shell life. As a result, it does not shrink due to evaporation of water, for example, as do the known agar or acrylic surfaces, which are disadvantageous in this regard.

It is particularly advantageous if the shell life of the elastomer is at least one year. The shell life shall be understood as the time window in which the apparatus, after production thereof, can be stored until use without the elastomer changing.

The elastomer in the apparatus according to the invention can advantageously be provided in a sterilized fashion. The elastomer is not influenced by sterilization, for example by gamma or UV radiation.

The layer thickness of the elastomer in the cell culture apparatus can range between about one hundred nanometers and several centimeters.

The layer thickness of the elastomer, and the thickness of the transparent apparatus, should advantageously be adjusted to each other and selected such that the cells can be examined by means of a microscope.

Thus, to be penetrated by most optical microscope lenses, the overall layer thickness of the apparatus and elastomer should not exceed approximately 250 μm.

Numerous different chemical substances from different groups are available as elastomers and can be used according to the invention for the surface of the cell culture apparatus. By way of example only, and without limiting the invention in any way, elastomers from the groups of siloxane resins, butadiene resins, and acrylate resins shall be mentioned. These resins have slightly hydrophobic properties and are generally suited for the cell culture method according to the invention.

The resins routinely comprise reactive groups, which form bonds with each other and thus can result in cross-linking the elastomer.

Below, the term base substance is used synonymously with the main component in the polymerized elastomer.

In the case of siloxane resins and butadiene resins, the reactive groups are present in shorter or longer non-reactive chain segments. The reactive groups of the siloxane resins, like the reactive groups of the butadiene resins, can form bonds with each other, without requiring the use of a copolymer. Copolymers additionally cross-link the reactive groups of the siloxane and butadiene resins with themselves.

Acrylate resins only comprise reactive groups which are cross-linked with themselves, either alone or with the addition of copolymers.

The base substance of the elastomer thus comprises monomer, oligomer, or polymer components, which are polymerized into higher-level structures by cross-linking.

Different techniques can be considered for cross-linking the elastomer components, which is to say the base substance and cross-linking agent (copolymer). Below, the terms cross-linking agent and copolymer are used synonymously.

Cross-linking is controlled, for example, by suitable copolymers, by UV light, ozone or, for example, by platinum catalysts at elevated temperatures. High temperatures typically increase the reaction speed during cross-linking.

Catalyst-driven polymerization processes are fast and efficient, and are performed, for example, for siloxane resins, using a platinum catalyst, such as by way of platinum divinyltetramethyldisoloxane (SIP6830.0 from ABCR “a better choice for chemical reagents”).

Resins, the base components of which cross-link by themselves when exposed to air at room temperature without the addition of a copolymer, are also available and can be used according to the invention.

The longer the selected chain length of the base substance (non-reactive chain segment) of the elastomer according to the invention is, the higher is the viscosity is, which means that the elasticity of the elastomer is proportionately lower after adding a suitable cross-linker.

Vice versa, it is accordingly true that, with decreasing chain length of the elastomer, elasticity increases after cross-linking the elastomer.

With respect to elasticity, any elastomer can be precisely and reproducibly adjusted through the suitable selection of factors, such as the chain length of the base substance, the mixing ratio of the base substance to the cross-linking agent (copolymer), or the addition of diluents.

Prior to polymerization, as compared to a resin comprising monomer components, the macromolecular structure of the base substance of siloxane resins having polymerizable terminal groups brings about a decrease in the speed of the diffusion of the reactive species, for example the vinyl groups, and of the concentration thereof. These disadvantages can be eliminated by lowering the viscosity, for example using reactive diluents, and by adding cross-linking agents.

Reactive diluents have a dual function. In addition to lowering the viscosity of the starting product, they also bring about an increase in the number of reactive groups per unit of volume of resin.

Elastomers of the group of butadiene resins are suitable for a cross-linking reaction, equivalent to the vulcanization of rubber using sulfur. These are resins in which the main chains of the oligomer components serving as the base substance are cross-linked via reactive sites, which are distributed throughout the entire chain, by monomer units of a cross-linking agent.

These oligomer chains can be linked to each other directly, without adding a cross-linking agent. The high viscosity of these oligomers, however, can also be reduced by reactive diluents and cross-linking agents.

Elastomers formed by the group of acrylate resins are preferably based on monomers as the base substance of the resin component. The resin comprises freely movable reactive groups. In this way, low viscosity, with good diffusion and a high concentration of reactive groups, can be achieved.

For elastomers comprising siloxane resins, the mixing ratio of the base substance to a cross-linking agent ranges from very high concentrations of cross-linking agents, to achieve low elasticity, or a high Young modulus (>1 MPa), to mixing ratios having low concentrations of cross-linking agents, to achieve high elasticity.

Elastomers having particularly good properties for the cell culture include mixtures of a vinyl-terminated siloxane as the base substance, and a methylhydrosiloxane-dimethylsiloxane copolymer as the cross-linking agent. Following cross-linking, the PDMS can be used as the surface for the cell culture.

Other siloxanes, such as hydroxysiloxanes comprising chlorosilane, are cross-linked directly with themselves, or by adding a copolymer, and can also be used within the scope of the invention as substrates for the cell culture.

Particularly advantageous PDMS elastomers as defined by the invention are formed by a base substance comprising oligomers having a molecular weight between 100 and 200,000.

A particularly suitable PDMS elastomer is produced from the Sylgard-184 kit (Dow Coming). The kit comprises vinyl-terminated siloxane resin as the base substance, and methylhydrosiloxane-dimethylsiloxane copolymer as the cross-linking agent in separate units, which are mixed with each other in suitable mixing ratios, depending on the desired elasticity, and polymerized.

DMS-V31 as the base substance for a vinyl-terminated polydimethyl siloxane (ABCR) and HMS 301 as an example of a methylhydrosiloxane-dimethylsiloxane copolymer for the cross-linking agent (ABCR) are also excellently suited to form elastomers for the cell culture.

The PDMS base components, which is to say the vinyl-terminated siloxane DMS-V31 and the methylhydrosiloxane-dimethylsiloxane copolymer serving as the cross-linking agent (HMS-301), are both liquid at room temperature. The polymerization process preferably occurs in the presence of a platinum catalyst, preferably at approximately 60° C.

By way of example, DMS-T21 (ABCR) as a polydimethylsiloxane shall be mentioned as a diluent for PDMS. It is not reactive, because no methylhydrosiloxane groups are present. It has a viscosity of 100 cSt. It can be admixed to PDMS that has not yet been cross-linked in an amount of up to 30 percent by volume.

By way of example, DMS-T22 (ABCR) as a polydimethylsiloxane shall also be mentioned as a diluent for PDMS; it is not reactive because no methylhydrosiloxane groups are present. It has a viscosity of 200 cSt. It can be admixed to PDMS that has not yet been cross-linked in an amount of up to 30 percent by volume.

The PDMS elastomers and the elastomers based on butadiene and acrylate resin can be applied with reproducibly even thickness onto the surface of the cell culture apparatus in an easy and inexpensive manner.

A person skilled in the art will consult a suitable reference for the reproducible production of elastomers.

In this respect, the following references and home pages shall be cited, the contents of which are hereby included in this application, particularly with respect to the elastomers, cross-linking agents and diluents stated therein:
W. Noll, Chemistry and Technology of Silicones, Academic Press, New York (1968).
T. C. Kendrick, B. Parbhoo, J. W. White, “Siloxane Polymers and Copolymers,” in The Chemistry of Organic Silicon Compounds Pt2 (edited by S. Patai and Z. Rappoport), 21, p. 1289-1361, John Wiley, Chichester (1989).
S. J. Clarson, J. A. Semlyen, Siloxane Polymers, Prentice Hall, New Jersey (1993).
J. W. White, R. C. Treadgold, “Organofunctional Siloxanes,” in Siloxane Polymers (edited by S. J. Clarson and J. A. Semlyen), 4, p. 193-215, Prentice Hall, New Jersey (1993).
W. Gardiner, J. W. White, “Specialty Silicones as Building Blocks for Organic Polymer Modification,” in High Value Polymers (edited by A. H. Fawcett), Royal Society of Chemistry, Cambridge (1990).
M. Brook, Silicon in Organic, Organometallic and Polymer Chemistry, John Wiley and Sons, New York (2000).
Dow Corning, home page, Silanes selection guide
ABCR, home page, Siloxane product list
M. Heger, Entwicklung eines Stereolithographieharzes für elastomere Produkte (Development of a stereolithography resin for elastomer products), Ph. D. thesis, Darmstadt, Germany (2001).

For siloxane resins that are composed of a base substance and a cross-linking agent, depending on the position of the reactive groups, which is to say whether this is a terminal position or within the chain, all known siloxane groups can be used both as the base substance or as the cross-linking agent.

Only by way of example, and without limiting invention in any way, only a few important elastomers of different groups shall be summarized below for the cell culture apparatus according to the invention:

Elastomers from the group of vinyl-functionalized siloxanes can be cross-linked with themselves by using peroxides or by adjusting the temperature. However, they can also be cross-linked with hydride-functionalized siloxanes by way of suitable selection of platinum catalysts.

Elastomers from the group of hydride-functionalized siloxanes are cross-linked with vinyl-functionalized siloxanes, optionally with platinum catalysts. However, they can also be cross-linked with silanol-functionalized siloxanes by means of metal salt catalysts.

Elastomers from the group of silanol-functionalized siloxanes can be cross-linked with hydride-functionalized siloxanes by way of metal salt catalysts. However, they can also be cross-linked with themselves by way of vulcanization at room temperature. Furthermore, they can be cross-linked with amino-functionalized siloxanes.

Elastomers from the groups of amino-functionalized siloxanes, epoxy-functionalized siloxanes, and carbinol-functionalized siloxanes are likewise suitable siloxanes as defined by the invention, that is, they are suitable as substrates for cells in cell culture apparatuses.

Elastomers from the group of methacrylate/acrylate-functionalized siloxanes can be cross-linked with themselves by means of radical generators, including UV light.

Elastomers from the group of mercapto-functionalized siloxanes can likewise be cross-linked with themselves, or also with vinyl-functionalized siloxanes by means of radical generators, including UV light.

Elastomers from the group of chorine/dimethylamine-functionalized siloxanes (α,ω-(methacryloxypropyldimethylsilyl)-polydimethylsiloxanes; methacryloxypropyldimethylchlorosilanes) are cross-linked with hydride-functionalized siloxanes by means of hydrolization of the chlorosilane bond.

By way of example, and without limiting the invention, possible diluents for the elastomers are medium to short chain length polydimethylsiloxanes having no reactive groups, or also siloxanes having reactive groups, but not involved in the cross-linking reaction used.

By way of example, and without limiting the invention, butadiene resins defined by the invention as the base substance may be carboxy-terminated oligobutadienes.

Butadiene resins used as cross-linking agents, or diluents, are, for example, hexanediol diacrylates, cyclohexyl methacrylates, methylacrylates, ethyleneglycol dimethacrylates, polyethyleneglycol monomethacrylates, linseed oil, styrenes, stearyl methacrylates, 2-hydroxyethy lacrylates, copolymers comprising n-butylacrylates and t-butylacrylates, n-hexylmethacrylates, 2-dimethylaminoethyl methacrylates, and n-decyl methacrylates.

An acrylate resin used as the base substance can be, for example, 2-hydroxypropylacrylate.

Acrylate resins used as cross-linking agents, or diluents, are, for example, n-hexylmethacrylates, ethyleneglycol dimethacrylates, and n-decyl methacrylates.

Elastomers, copolymers, and diluents, which are encompassed by the above groups and/or disclosed in the references that have been incorporated, are expressly encompassed by the idea of the invention.

It is possible to form a plurality of elastomers that vary slightly from each other using only a single combination of the base substance and cross-linking agent, for example, the Sylgard-184 kit. In this way, the Young modulus can be varied within the range mentioned above, for example, by changing the mixing ratio of the base substance to the cross-linking agent.

This also applies, as a matter of course, to the remaining elastomers mentioned above and in the references. Such minor changes are likewise expressly encompassed by the idea of the invention.

An unstructured surface comprising an elastomer can be very quickly applied to, or produced on, the surface of a suitable cell culture apparatus using a suitable method. In this way, advantageous industrial mass production of the cell culture apparatus according to the invention is possible using the known cell culture apparatuses.

A uniform layer of the elastomer covering the cell culture apparatus can be adjusted virtually arbitrarily by a person skilled in the art, if a suitable method, such as spin coating or a potting method is employed.

When mixtures of base substance and optional copolymer (cross-linking agent) are employed, layer thicknesses of approximately 100 nanometers up to several centimeters can be reproducibly manufactured. To this end, the base substance, and optionally a cross-linking agent and/or catalyst, are mixed and uniformly distributed in, or on, the cell culture apparatus, so that the surface, which is provided in order to receive the cell, is formed by the unstructured elastomer.

The cross-linking process for obtaining the elastomer normally occurs only after the elastomer has been applied to the cell culture apparatus.

In order to produce highly elastic surfaces for the cell culture, advantageously a method should be employed, whereby an unstructured, planar elastomer film can be reproducibly produced quickly on the surface of the apparatus on an industrial scale. This allows mass production of the cell culture apparatuses according to the invention.

Spin methods are particularly advantageous for the production of very thin and uniform layers of the elastomer.

Here, a small amount of a liquid elastomer that has not yet been cross-linked is added to the surface of the apparatus and distributed by means of a centrifugal force using a suitable apparatus that performs rotation.

In the case of elastomers comprising a cross-linking agent, this agent is added in advance to the base substance and mixed therewith, and a catalyst is optionally added.

The apparatus, together with the liquid elastomer, is clamped into a spin coater, for example. Alternatively, however, the apparatus can also be held on a rotary table by means of a vacuum.

By way of rotation, for example at 1000 rpm per minute, the elastomer is distributed on the bottom of the cell culture vessel as a very uniform unstructured film. The resulting layer is then used to receive the cells in the cell culture method.

After curing, which may optionally be driven by a catalyst, at least one elastomer from the group of siloxane resins maintains the elastic properties thereof for a nearly unlimited time and without additional work.

The resulting layer thickness depends on the viscosity and the amount the elastomer, which has not yet been cross-linked, as well as the rotational speed, and the acceleration and process duration during spin coating.

A person skilled in the art can reproducibly form a variety of layer thicknesses of elastomers when using the described process parameters.

For example, it is possible to regularly apply a small amount of the uncross-linked elastomer to the surface of a desired apparatus, and distribute it thereon, using approximately 1000 revolutions per minute, by means of a spin coater.

As an alternative to the spin method, it is possible to pot the elastomer, without subsequent rotation, or use a brush method, wherein uniform layer thicknesses are applied to surfaces by means of a suitable apparatus, or a paint roller method, wherein the elastomer is rolled onto surfaces by means of a paint roller, or a rolling method, or one may use a combination of the different methods above, as a conceivable alternative to the spin method.

In particular, Petri dishes, microscope slides, or cover slips, having a corresponding surface that faces the cell culture, can be produced from the above elastomers. In addition, multi-well plates, cell culture bottles, and other shapes, which are common according to the state of the art for cell culture, can be uniformly coated with an elastomer.

The idea of the invention can be applied to any cell culture vessel, or any cell culture apparatus, known to date.

As a result, a person skilled in the art can coat any cell culture apparatus disclosed in the relevant laboratory and technical catalogs with a surface comprising an elastomer. In this way, a new category of cell culture vessels and apparatuses is provided in a particularly advantageous manner.

A step of washing the cross-linked elastomer using isopropanol, and particularly pure isopropanol, increases the transparency of the elastomer.

This is extremely important for an unstructured planar surface. In general, a step of washing in alcohol lasting approximately 3 hours is sufficient.

Particularly in the case of higher elasticity for the elastomer of the apparatus according to the invention, having a Young modulus of <50 kPa, a washing step using isopropanol ensures that the uncross-linked components do not diffuse out of coalesced bubbles in the layer. The washing step thus facilitates observation of the cells.

It is conceivable to use a different substance or alcohol.

In any case, the surface of the cell culture apparatus comprising the elastomer should preferably look like that of glass to ensure easier observation of the cells.

In a particularly advantageous embodiment of the invention, the elastomer for the cell culture apparatus can be present in activated form by physisorption of biomolecules.

When using physisorption, special adhesion proteins are applied to the surface of the apparatus. Physisorption generally denotes a passive surface coating process, during which biomolecules from an aqueous solution deposit on the surface beneath and build a more or less uniform layer. In this way, defined adhesion conditions for cells are specified, which enable the analysis of very specific questions. By way of example only, the analysis of biochemical aspects of cell-substrate interactions under defined conditions shall be mentioned.

For this purpose, biomolecules, such as fibronectin, laminin, collagen, tenascin, or hyaluronic acid, are first dissolved in a buffer. The liquid, together with the biomolecule, is applied to the previously cross-linked elastomer. In the process, the biomolecule passively adheres to the elastomer as a thin layer. The residual fluid is then removed.

The biomolecule for any arbitrary elastomer is selected substantially on the basis of the experiment or on the basis of the cells.

In this way, it is particularly advantageously possible to at least partially eliminate the hydrophobic property of the elastomer, and simultaneously specific interactions are enabled under in vivo conditions between the protein, which serves as the biomolecule, and the cells. As a result, the elastomers are particularly well-suited for cell culture methods.

However, it is also conceivable to perform the cell culture method according to the invention in an apparatus according to the invention without such changes, since some cell types do not require elastomers that have been modified by physisorption.

As described in the example, with respect to the differentiation of cells after the addition of therapeutics, the many possible combinations in terms of elastomer parameters, elasticity, and the type of physisorption present numerous possibilities for handling specific experiments, which cannot be pursued with existing cell culture methods.

It is conceivable to dispose separate cross-members as structures in the elastomer for complex experiments in order to detect the reaction of the cells on chemicals or therapeutic agents. It is thus possible to analyze chemotaxis in the broadest sense, as well as division behavior under these conditions.

It is conceivable to arrange more complex layer sequences, for example by printing biomolecules by means of microcontact or nanocontact printing, after prior passivation of the intermediate regions.

A cell culture method according to the invention provides for the application of cells, for example from a starter culture, onto an optionally modified elastomer of the apparatus and the cultivation thereof.

The term cell culture is very broad with respect to the cells used. It comprises procaryotic and eucaryotic, isolated cells, as well as multi-cell structures. The apparatuses according to the invention are particularly advantageous for cells of eucaryotic origin because these cells generally adhere.

The term cell culture also comprises the analysis of cells in active ingredient research under nearly natural elasticity. To this end, the differentiation, division rates and/or migration behavior of the cells on the elastomer are of particular interest.

Depending on the cell type used, the necessary framework conditions for the culture and for the elastomer must be adjusted.

By way of example, the selection of a suitable synthetic, liquid culture medium, adjuvants such as vitamins, and the temperature or the shape of the culture vessel shall be mentioned as framework conditions.

In a particularly advantageous embodiment of the invention, the differentiation of the cell can be specifically controlled during the cell culture method by way of the Young modulus of the elastomer.

Advantageously, fibroblasts that were applied to the elastomer can be differentiated into different cell types, such as cardiac fibroblasts or myofibroblasts, as a function of the Young modulus of the elastomer. This, in turn, is of interest in the analysis of active ingredients and the provision of new therapeutic agents.

It was recognized as part of the invention that, as a measure for the division rate of cells, the number of cell divisions per unit of time can also be controlled via the Young modulus of the elastomer surface of the cell culture vessel. Depending on the cell type used, this increases by as much as 40% on soft surfaces, as compared with apparatuses according to the state of the art.

The invention will be described in more detail below with reference to embodiments and the accompanying figures.

### BRIEF DESCRIPTION OF THE DRAWINGS

FIG. 1 is a schematic representation of the cross-linking types of several resins (state of the art) for cell culture apparatuses according to the invention;

FIG. 2a depicts an antibody labeling of the cytoskeleton of adhereing fibroblasts after a six-day culture in a Petri dish made of polypropylene (state of the art) comprising a centrally disposed cover slip as the cell culture bottom;

FIG. 2b depicts a transmitted light print corresponding to FIG. 2a;

FIG. 3a depicts an antibody labeling of the cytoskeleton of adhereing fibroblasts after a six-day culture in a Petri dish made of polypropylene comprising a centrally disposed cover slip and an elastic surface disposed thereon, comprising PDMS, having a Young modulus of 38 kPa, as the cell culture bottom;

FIG. 3b depicts a transmitted light print corresponding to FIG. 3a; and

FIG. 4 is a confocal side view of a cell culture apparatus according to FIG. 3.

### DETAILED DESCRIPTION OF THE INVENTION

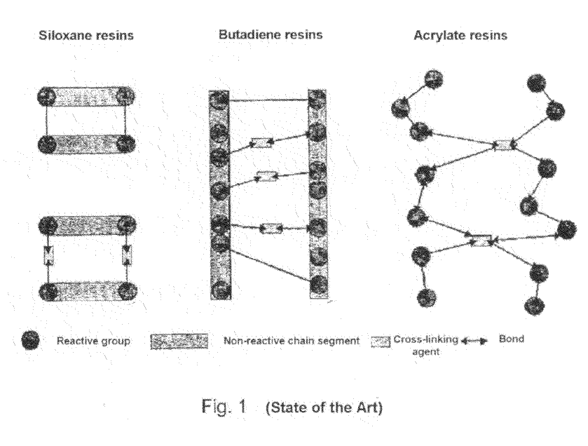

FIG. 1 schematically shows the cross-linking types of three kinds of resins, which can be used according to the invention as the unstructured substrate for cells (state of the art).

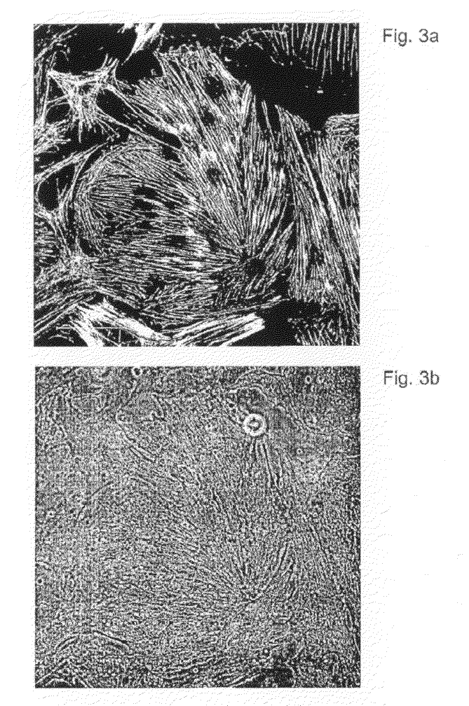

For the arrangement according to FIG. 3, fibroblasts were isolated and plated out onto a PDMS surface, made from a Sylgard-184 kit, at a concentration of approximately 5000 cells per cm².

The kit comprises vinyl-terminated siloxane as the base substance, a methylhydrosiloxane-dimethylsiloxane as the copolymer and an admixed Pt catalyst.

The mixture comprising siloxane and copolymer was prepared at a mixing ratio of 50:1 of base substance to cross-linking agent, mixed, and degassed in a desiccator.

A cover slip measuring 25×75 mm and having a thickness of approximately 100 μm was mounted on the rotary table of a Delta 10T type spin coater, from Süss Microtech, and coated with 3 ml of PDMS, which was not yet cross-linked. Spinning was performed for 30 seconds at 1000 revolutions per minute.

The bottom of the cover slip was then cleaned of excess PDMS using n-heptane. Curing occurred in dustproof receptacles at 60° C., overnight, achieving elasticity of approximately 25 kPa.

After cross-linking, the elastomer was coated with 2.5 μg/cm² of fibronectin as an additional layer for promoting adhesion of the cells. Fibronectin ensures adhesion of the cells under nearly natural and defined conditions. For this purpose, an appropriately small amount was applied and the residual fluid was removed after 20 minutes.

The elastomer, was rinsed in 2-propanol for 15 hours under gentle agitation in order to wash out excess silicon oil, which was not cross-linked. In the PDMS polymer described, having a Young modulus of approximately 25 kPa, this brings about a 40% volume reduction, while simultaneously improving transparency, and brings about post-curing to approximately 38 kPa. The layer thickness of the elastomer that was produced amounted, after this step, to approximately 40 μm at a Poisson's ratio of approximately 0.5.

The cover slip was glued over the elastomer from beneath onto a centrally disposed hole in the bottom of a Petri dish. The bond between the PDMS surface and the cell culture vessel occurred directly via the adhesive properties of the PDMS, which was already cross-linked on the surface.

Alternatively, it is possible to increase adhesion by use of an additional cell-compatible adhesive.

As a comparison specimen, an identical cover slip comprising no such elastomer was glued to an otherwise identical Petri dish.

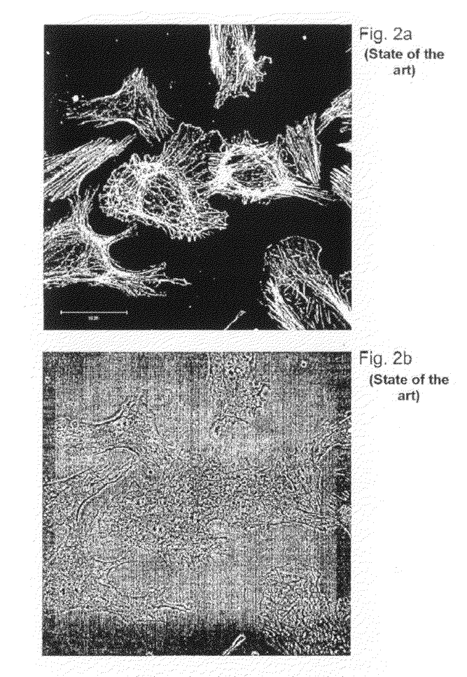

Embryonal myocardial fibroblasts were isolated and seeded as individual cells onto the elastomer (FIG. 3), or directly onto the glass surface of the cover slip (FIG. 2).

The cells according to FIG. 3 were incubated, as with the cells in FIG. 2, for six days at 37° C. in a suitable nutrient solution. Thereafter, the cells were fixed with formaldehyde and labeled against smooth muscle actin with an antibody and incubated. Smooth muscle actin is considered a marker for the nearly natural function of cellular energy generation. A person skilled in the art can easily obtain the corresponding fixation and labeling protocols from the corresponding technical literature.

The comparison between FIG. 2a and FIG. 3a shows that, after 6 days of culture, only the fibroblasts on PDMS (FIG. 3a) had formed a clearly defined, dense and parallel oriented cytoskeleton. On the substrate of the comparison specimen formed by the cover slip (FIG. 2a), this structure of parallel orientation of the cytoskeleton, which is absolutely essential for the function thereof, was not formed.

The corresponding transmitted light prints show that, on the PDMS surface (FIG. 3b), the cell count was approximately 40% higher than on the glass surface (FIG. 2b). This demonstrates the increased cell division rate on PDMS, as compared to glass, and indicates considerably lower cell stress when using an elastic surface.

The experiment illustrates that the morphology of the cells, and therefore the structure and function thereof, is positively influenced when selecting an elastomer as the substrate. The same applies to the cell count.

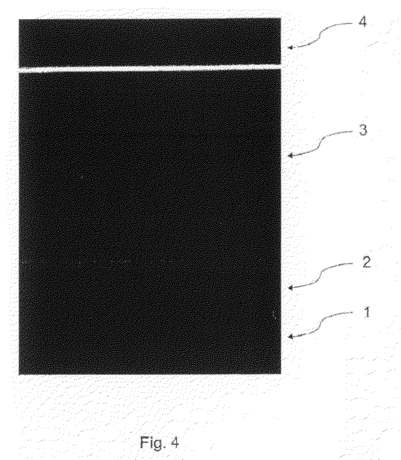

The resulting layer thickness of the elastomer 3 is approximately 40 μm, as is shown in FIG. 4. Therein, layer 4 shows the adjoining air, layer 2 constitutes the cover slip, and layer 1 is the fluid disposed on the PDMS.

1.-36. (canceled)

37. A cell culture apparatus, comprising:
an apparatus support structure; and
a layer comprised of an elastomer carried on said apparatus support structure, said layer having an unstructured surface for receiving cells.

38. A cell culture apparatus according to claim 37, wherein said layer is configured as a thin film.

39. A cell culture apparatus according to claim 37, wherein said elastomer is a transparent elastomer.

40. A cell culture apparatus according to claim 37, wherein the elastomer has a refractive index in a range of about 1.3 to 1.5.

41. A cell culture apparatus according to claim 37, wherein the elastomer has a refractive index of about 1.4.

42. A cell culture apparatus according to claim 37, wherein said elastomer has a Young modulus of 1 MPa to 500 Pa.

43. A cell culture apparatus according claim 37, wherein a surface of the elastomer comprises a layer promoting the adhesion and culture of the cells.

44. A cell culture apparatus according to claim 43, wherein said layer comprises at least one of fibronectin, laminin, collagen, tenascin, or hyaluronic acid.

45. A cell culture apparatus according to claim 37, wherein said elastomer has a Poisson's number in a range of about 0.3 to 0.5.

46. A cell culture apparatus according to claim 37, wherein said elastomer is not water-based.

47. A cell culture apparatus according to claim 37, wherein said elastomer comprises a base substance that includes a siloxane resin, a butadiene resin, or an acrylate resin.

48. A cell culture apparatus according to claim 37, wherein said elastomer comprises a base substance that includes a vinyl-terminated siloxane resin.

49. A cell culture apparatus according to claim 37, wherein said elastomer comprises a methylhydrosiloxane-dimethylsiloxane copolymer as a cross-linking agent.

50. A cell culture apparatus according to claim 37, wherein said apparatus support structure comprises at least a portion of at least one of a Petri dish, a microscope slide, a cover slip, a multi-well plate, a cell culture bottle, a sample carrier for the analysis of chemical or biological samples.

51. A cell culture apparatus according to claim 37, wherein said apparatus support structure comprises a sample carrier comprising cross-members and channels of a microfluid system or a cell culture chamber.

52. A cell culture apparatus according to claim 37, wherein said elastomer comprises a suitable material for in situ hybridization and/or antibody labeling of the cells.

53. A method for producing a cell culture apparatus, comprising:
coating a surface of an apparatus support structure for receiving cells with an elastomer to form a layer having an unstructured surface.

54. A method according to claim 53, wherein said layer is formed as a thin film.

55. A method according to claim 53, wherein a base substance of said elastomer is selected from the group consisting of siloxane resin, butadiene resin, or acrylate resin.

56. A method according to claim 53, wherein a base substance of said elastomer is a vinyl-terminated siloxane resin.

57. A method according to claim 53, wherein a base substance of said elastomer is a chloride-terminated siloxane resin.

58. A method according to claim 55, further comprising cross-linking a resin which comprises a base substance of said elastomer by mixing said resin with a copolymer as a cross-linking agent before said coating the surface of the apparatus support structure.

59. A method according to claim 58, wherein said mixing includes mixing said resin and said copolymer with a catalyst.

60. A method according to claim 58, wherein a transparent elastomer is formed.

61. A method according to claim 53, wherein said coating the surface of the apparatus support structure includes performing a spin method.

62. A method according to claim 53, wherein said coating the surface of the apparatus support structure includes performing a brush method, a paint roller method, or a potting method.

63. A method according to claim 53, wherein said cross-linking is carried out at a temperature of about 60° to 120° Celsius.

64. A method according to claim 53, further comprising polymerizing the elastomer under UV light.

65. A method according to claim 53, further comprising:
polymerizing the elastomer to form a polymerized elastomer; and
washing the polymerized elastomer with isopropanol.

66. A method according to claim 53, further comprising:
curing the elastomer; and
sterilizing the apparatus after the curing.

67. A method according to claim 53, further comprising producing the elastomer with a Young modulus of 1 MPa to 500 Pa.

68. A method of cell culture, comprising:
providing a cell culture apparatus including an elastomer having an unstructured surface; and
cultivating cells in said cell culture apparatus on said unstructured surface.

69. A method of cell culture according to claim 68, wherein the cultivating of the cells is carried out on a siloxane resin, butadiene resin, or acrylate resin.

70. A method of cell culture according to claim 68, wherein the cultivating includes controlling at least one of a differentiation, a division rate or a migration behavior of the cells by way of a Young modulus of the elastomer.

71. A method of cell culture according to claim 68, wherein the cultivating includes adding therapeutic agents.

72. A method of cell culture according to claim 68, wherein the cultivation includes cultivation of fibroblasts on the elastomer.

73. A method of cell culture according to claim 68, wherein a kit comprising PDMS is used as the elastomer.

74. A method of cell culture according to claim 73, wherein said PDMS is produced from a Sylgard-184 kit.

75. A method of cell culture according to claim 68, wherein said cultivating includes cultivation of the cells on the elastomer having a Young modulus of 1 MPa to 500 Pa.

76. A method of cell culture according to claim 68, wherein:
said elastomer is a transparent elastomer; and
said cultivating includes cultivation of the cells on said transparent elastomer.

77. A method of cell culture according to claim 68, wherein;
said elastomer includes a layer promoting adhesion and culture of the cells; and
said cultivating includes cultivation of the cells on said layer promoting the adhesion and culture of the cells.

78. A method of cell culture according to claim 77, wherein said layer promoting the adhesion and culture of the cells comprises fibronectin, laminin, collagen, tenascin, or hyaluronic acid.
